SEQUENCE LISTING (62 sequences)

SEQ ID NO: 1 - protein, 17 aa, Artificial Sequence
  ApoE mimetic peptide
LRVRLASHLRKLRKRLL

SEQ ID NO: 2 - protein, 34 aa, Artificial Sequence
  ApoE mimetic peptide
RQIKIWFQNRRMKWKKCLRVRLASHLRKLRKRLL

SEQ ID NO: 3 - protein, 17 aa, Artificial Sequence
  ApoE mimetic peptide
LLRKRLKRLHSALRVRL

SEQ ID NO: 4 - protein, 12 aa, Artificial Sequence
  ApoE mimetic peptide
ASXLRKLXKRLL

SEQ ID NO: 5 - protein, 17 aa, Artificial Sequence
  ApoE mimetic peptide
LRVRLASXLKRLXKRLL

SEQ ID NO: 6 - protein, 17 aa, Artificial Sequence
  ApoE mimetic peptide
LRVRLASXLRKLRKRLL

SEQ ID NO: 7 - protein, 17 aa, Artificial Sequence
  ApoE mimetic peptide
RQIKIWFQNRRMKWKKC

SEQ ID NO: 8 - protein, 27 aa, Artificial Sequence
  ApoE mimetic peptide
RRLSYSRRRFLRVRLASHLRKLRKRLL

SEQ ID NO: 9 - protein, 13 aa, Unknown
  Protein transduction domain peptide
GRKKRRQRRRPPQ

SEQ ID NO: 10 - protein, 16 aa, Unknown
  Protein transduction domain peptide
RQIKIWFQNRRMKWKK

SEQ ID NO: 11 - protein, 7 aa, Unknown
  Protein transduction domain peptide
RRMKWKK

SEQ ID NO: 12 - protein, 18 aa, Unknown
  Protein transduction domain peptide
RGGRLSYSRRRFSTSTGR

SEQ ID NO: 13 - protein, 10 aa, Unknown
  Protein transduction domain peptide
RRLSYSRRRF

SEQ ID NO: 14 - protein, 17 aa, Unknown
  Protein transduction domain peptide
RGGRLAYLRRRWAVLGR

SEQ ID NO: 15 - protein, 8 aa, Unknown
  Protein transduction domain peptide
RRRRRRRR

SEQ ID NO: 16 - protein, 17 aa, Artificial Sequence
  ApoE mimetic peptide
LRVRLASHXRKLRKRLL

SEQ ID NO: 17 - protein, 12 aa, Artificial Sequence
  ApoE mimetic peptide
ASHXRKLRKRLL

SEQ ID NO: 18 - protein, 12 aa, Artificial Sequence
  ApoE mimetic peptide
ASXLRKLRKRLL

SEQ ID NO: 19 - protein, 12 aa, Artificial Sequence
  ApoE mimetic peptide
DSXLRKLRKRLL

SEQ ID NO: 20 - protein, 12 aa, Artificial Sequence
  ApoE mimetic peptide
ASHLRKLXKRLL

SEQ ID NO: 21 - protein, 15 aa, Artificial Sequence
  ApoE mimetic peptide
DRXASHLRKLRKRXL

SEQ ID NO: 22 - protein, 12 aa, Artificial Sequence
  ApoE mimetic peptide
DSXLRKLRKRXL

SEQ ID NO: 23 - protein, 15 aa, Artificial Sequence
  ApoE mimetic peptide
DRXASHLRKLXKRLL

SEQ ID NO: 24 - protein, 12 aa, Artificial Sequence
  ApoE mimetic peptide
DSXLRKLXKRLL

SEQ ID NO: 25 - protein, 15 aa, Artificial Sequence
  ApoE mimetic peptide
DRXASXLRKLRKRLL

SEQ ID NO: 26 - protein, 15 aa, Artificial Sequence
  ApoE mimetic peptide
DRXASHLRKLRKRLL

SEQ ID NO: 27 - protein, 13 aa, Artificial Sequence
  ApoE mimetic peptide
CASXLRKLXKRLL

SEQ ID NO: 28 - protein, 12 aa, Artificial Sequence
  ApoE mimetic peptide
DSXLRKLXKRLL

SEQ ID NO: 29 - protein, 12 aa, Artificial Sequence
  ApoE mimetic peptide
ASXLRKLXKRLV

SEQ ID NO: 30 - protein, 12 aa, Artificial Sequence
  ApoE mimetic peptide
ASXLRKLXKRLM

SEQ ID NO: 31 - protein, 12 aa, Artificial Sequence
  ApoE mimetic peptide
ASXLRKLXKRLI

SEQ ID NO: 32 - protein, 12 aa, Artificial Sequence
  ApoE mimetic peptide
ASXLRKLXKRLA

SEQ ID NO: 33 - protein, 12 aa, Artificial Sequence
  ApoE mimetic peptide
ASXLRKLXKALL

SEQ ID NO: 34 - protein, 12 aa, Artificial Sequence
  ApoE mimetic peptide
ASXLRKLXKXLL

SEQ ID NO: 35 - protein, 12 aa, Artificial Sequence
  ApoE mimetic peptide
ASXLRKLXKXLL

SEQ ID NO: 36 - protein, 12 aa, Artificial Sequence
  ApoE mimetic peptide
ASXLRKLXKXLL

SEQ ID NO: 37 - protein, 12 aa, Artificial Sequence
  ApoE mimetic peptide
ASXLRKLXKXLL

SEQ ID NO: 38 - protein, 12 aa, Artificial Sequence
  ApoE mimetic peptide
ASXLRKLXARLL

SEQ ID NO: 39 - protein, 12 aa, Artificial Sequence
  ApoE mimetic peptide
ASXLRKLXXRLL

SEQ ID NO: 40 - protein, 12 aa, Artificial Sequence
  ApoE mimetic peptide
ASXLRKLXXRLL

SEQ ID NO: 41 - protein, 12 aa, Artificial Sequence
  ApoE mimetic peptide
ASHXRKLXKRLL

SEQ ID NO: 42 - protein, 12 aa, Artificial Sequence
  ApoE mimetic peptide
ASXLRKLXKRLX

SEQ ID NO: 43 - protein, 12 aa, Artificial Sequence
  ApoE mimetic peptide
ASXLRKLXKRXL

SEQ ID NO: 44 - protein, 12 aa, Artificial Sequence
  ApoE mimetic peptide
ASXLRKLXKRXX

SEQ ID NO: 45 - protein, 12 aa, Artificial Sequence
  ApoE mimetic peptide
ASXLRKLXKXLX

SEQ ID NO: 46 - protein, 12 aa, Artificial Sequence
  ApoE mimetic peptide
ASXLRKLXKXXL

SEQ ID NO: 47 - protein, 12 aa, Artificial Sequence
  ApoE mimetic peptide
ASXLRKLXKXXX

SEQ ID NO: 48 - protein, 12 aa, Artificial Sequence
  ApoE mimetic peptide
ASXLRKLXKXLX

SEQ ID NO: 49 - protein, 12 aa, Artificial Sequence
  ApoE mimetic peptide
ASXLRKLXKXXL

SEQ ID NO: 50 - protein, 12 aa, Artificial Sequence
  ApoE mimetic peptide
ASXLRKLXKXXX

SEQ ID NO: 51 - protein, 12 aa, Artificial Sequence
  ApoE mimetic peptide
ASXLXKLXKRLL

SEQ ID NO: 52 - protein, 12 aa, Artificial Sequence
  ApoE mimetic peptide
ASXLXKLXKXLL

SEQ ID NO: 53 - protein, 12 aa, Artificial Sequence
  ApoE mimetic peptide
ASXLXKLXKRLX

SEQ ID NO: 54 - protein, 13 aa, Artificial Sequence
  ApoE mimetic peptide
ASXLXKLXKRLXX

SEQ ID NO: 55 - protein, 12 aa, Artificial Sequence
  ApoE mimetic peptide
ASXLXKLXKXLX

SEQ ID NO: 56 - protein, 12 aa, Artificial Sequence
  ApoE mimetic peptide
ASXLXKLXKXXX

SEQ ID NO: 57 - protein, 12 aa, Artificial Sequence
  ApoE mimetic peptide
ASHLRKLRKRLL

SEQ ID NO: 58 - protein, 12 aa, Artificial Sequence
  ApoE mimetic peptide
ASHCRKLCKRLL

SEQ ID NO: 59 - protein, 12 aa, Artificial Sequence
  ApoE mimetic peptide
ASCLRKLCKRLL

SEQ ID NO: 60 - protein, 12 aa, Artificial Sequence
  ApoE mimetic peptide
CSHLRKLCKRLL

SEQ ID NO: 61 - protein, 12 aa, Artificial Sequence
  ApoE mimetic peptide
ASHLRKCRKRCL

SEQ ID NO: 62 - protein, 12 aa, Artificial Sequence
  ApoE mimetic peptide
ASHCRKLRKRCL

(19) United States
(12) Patent Application Publication
(10) Pub. No.: US 2011/0166079 A1
(43) Pub. Date: Jul. 7, 2011

(54) METHODS OF TREATING CANCER WITH APOE PEPTIDES

(75) Inventors: Michael P. Vitek, Cary, NC (US); Dale J. Christensen, Cary, NC (US)

(73) Assignee: COGNOSCI, INC., Research Triangle Park, NC (US)

(21) Appl. No.: 13/002,247

(22) PCT Filed: Jul. 1, 2009
(86) PCT No.: PCT/US09/49389

§ 371 Date: Mar. 14, 2011

Related U.S. Application Data
(60) Provisional application No. 61/077,311, filed on Jul. 1, 2008.

(51) Int. Cl. A61K 38/16; A61K 38/00; A61K 38/03; A61P 35/00

(52) U.S. Cl. 514/19.4; 514/19.3; 514/19.6

(57) ABSTRACT

Methods of treating chronic lympocytic leukemia, chronic myelogenous leukemia, and breast cancer in a subject by administering an ApoE peptide are disclosed.

### CROSS-REFERENCE TO RELATED APPLICATIONS

This application claims priority to U.S. Provisional Application No. 61/077,311, filed Jul. 1, 2008, which is herein incorporated by reference in its entirety.

### FIELD OF THE INVENTION

The present invention relates to methods of treating cancer by administering at least one peptide derived from apolipoprotein E (ApoE). Administration of the ApoE peptides induces apoptosis of tumor cells and reduces tumor formation, tumor growth, and spread of tumor cells. In particular, methods of treating various types of leukemia and breast cancer are described.

### BACKGROUND OF THE INVENTION

Cancer is a class of diseases in which a group of cells exhibit uncontrolled growth, invasion and destruction of adjacent tissues, and metastasis (spread of aberrant cells spread to other locations in the body), or in which cells fail to undergo programmed cell death (e.g. apoptosis) at the appropriate time. Cancer causes about 13% of all deaths and according to the American Cancer Society, 7.6 million people died from cancer in the world during 2007. Current treatment for cancer depends upon the specific type of cancer and tissue involved, but includes surgery, chemotherapy, radiation therapy, immunotherapy, and monoclonal antibody therapy among other methods. Although these treatment methods have been successful in some cases, they are hindered by adverse side effects or limited efficacy. For example, the efficacy of eliminating cancerous tissue by surgical removal of tumors is often limited by the tendency of cancers to invade adjacent tissue and metastasize to other sites in the body. Chemotherapy, as well as radiation treatment, is often limited by toxicity or damage to other tissues in the body. Thus, cancer remains a major health concern and there is a need for improved methods of treating cancer.

Two of the most lethal common cancers in the United States for men and women are leukemia and breast cancer, respectively. There are four basic categories of leukemia: acute lymphocytic leukemia (ALL), acute myelogenous leukemia (AML), chronic lymphocytic leukemia (CLL), and chronic myelogenous leukemia (CML). CLL is the most frequent leukemia in the Western world with nearly 84,000 cases in the 7 largest industrialized nations. CLL is a malignant condition that is caused primarily by defects in the programmed cell death process known as apoptosis. As a result of dysregulated apoptosis, mature monoclonal B cells that easily disrupt during preparation of blood smears, accumulate in the blood (Rozman et al. (1995) N. Engl. J. Med., Vol. 333: 1052-1057). In CLL, the malignant cells are small B lymphocytes that are characterized by expression of most of the surface markers presented by mature B cells with some minor heterogeneity (Caligaris-Cappio and Janossy (1985) Semin Hematol., Vol. 22: 1-12). The most distinctive phenotypic features of malignant CLL cells are virtually undetectable amounts of monoclonal surface immunoglobulins and the expression of CD5, a surface marker that is normally found on mature T-cells but not on mature B-cells (Boumsell et al. (1978) Eur. J. Immunol., Vol. 8: 900-904; Ternynck et al. (1974) Blood, Vol. 43: 789-795). The clinical course of CLL is heterogeneous where some patients may experience an aggressive course that demands intensive treatment while others may experience a long survival without ever requiring treatment. The disease is progressive because the malignant clone acquires sequential genetic abnormalities that progressively increase its malignant behavior. CLL is most prevalent in older males and the median age at diagnosis is 64 years. Notably, CLL is the only adult leukemia that is not associated with exposure to ionizing radiation or chemicals and it does not occur in higher frequency in patients with immunodeficiency syndromes.

CML affects nearly 15,000 patients worldwide and is a disorder of the pluripotent hematopoietic stem cells with two distinct phases. The protracted myelopoliferative chronic phase is followed by a rapidly fatal blast crisis. In CML, a chromosomal translocation leads to production of a fusion between BCR protein and the Abl kinase that leads to constitutive activation of Abl. This constitutive activation of Abl has been shown to be sufficient for induction of chronic phase CML. Although progress has been made in treatment of CML with the introduction of Gleevec and other inhibitors of BCR/Abl, recently Gleevec resistant-CML has been reported and is a growing concern.

Given the number of different treatments for each specific type of cancer as well as the potentially severe side effects of such treatments, there is an existing need to develop novel cancer therapeutics that are effective for more than one type of cancer without causing unwanted toxicity or damage to healthy tissues.

### SUMMARY OF THE INVENTION

The present invention is based on the discovery that ApoE peptides can be used to treat cancer. Thus, the present invention provides a method of treating cancer in a subject in need thereof comprising administering an effective of amount of at least one ApoE peptide to the subject. In one embodiment, administration of said ApoE peptide decreases tumor formation in the subject. In another embodiment, administration of said ApoE peptide reduces tumor size in the subject. In another embodiment, administration of said ApoE peptide induces apoptosis of a cancer cell in the subject. In still another embodiment, administration of said ApoE peptide reduces the spread of cancer cells to healthy tissues in the subject.

The ApoE peptide may contain ten or more residues of the native ApoE holoprotein. In one embodiment of the invention, the ApoE peptide is COG133 (SEQ ID NO: 1). In another embodiment, the ApoE peptide is COG112 (SEQ ID NO: 2) or COG068 (SEQ ID NO: 8). In still another embodiment, the ApoE peptide is a COG133 derivative such as COG1410 (SEQ ID NO: 4) or COG345 (SEQ ID NO: 6). Other ApoE peptides useful in the present invention are described in U.S. Application Publication No. 2009/0042783 A1, which is herein incorporated by reference in its entirety.

In one embodiment, the ApoE peptide can contain SEQ ID NO: 1, SEQ ID NO: 2, or SEQ ID NO: 4 or any of the derivatives described in U.S. Application Publication No. 2009/0042783 A1 linked to one to five additional amino acids or amino acid analogs at the N-terminus or C-terminus or both the N-terminus and C-terminus, wherein such additional amino acids do not adversely affect the activity of the peptide. The ApoE peptide containing SEQ ID NO: 1 or SEQ ID NO: 2 or other ApoE derived peptide can contain 12 amino acids or more, 13 amino acids or more, 14 amino acids or more, 15 amino acids or more, 16 amino acids or more, 17 amino acids or more, 18 amino acids or more, 19 amino acids or more, 20 amino acids or more, 25 amino acids or more, 30 amino acids or more, 35 amino acids or more, or 40 amino acids or more. In some embodiments, the ApoE peptide consists essentially of SEQ ID NO: 1, SEQ ID NO: 2, or SEQ ID NO: 4. In other embodiments, the ApoE peptide is conjugated to a protein transduction domain to facilitate penetration of the cell. The protein transduction domain may include peptides derived from antennapedia, TAT, SynB1, SynB3, SynB5, and polyarginine.

The present invention also encompasses methods of treating leukemia in a subject in need thereof by administering an effective amount of at least one ApoE peptide. In one embodiment, said leukemia is chronic lymphocytic leukemia (CLL). In another embodiment, said leukemia is chronic myelogenous leukemia (CML). In some embodiments, administration of the ApoE peptide may decrease the number of CD5+ B cells in the subject. In other embodiments, administration of the ApoE peptide may decrease the growth of BCR/ABL+ cells in the subject. In certain embodiments, administration of the ApoE peptide can decrease the growth of imatinib- or dasatinib-resistant BCR/ABL+ cells in the subject.

The present invention also contemplates a method of treating breast cancer in a subject in need thereof. In one embodiment, the method comprises administering an effective amount of at least one ApoE peptide to the subject. In another embodiment, the breast cancer is characterized by Her2 expression. In another embodiment, the breast cancer is characterized by estrogen receptor expression.

### BRIEF DESCRIPTION OF THE DRAWINGS

FIG. 1. Schematics of aberrant signaling cascades in various forms of cancer. (A) Overactivation of the PI-3Kinase-Akt signaling cascade produces a persistent anti-apoptotic state in chronic myelogeneous leukemia (CML) and breast cancer. Constitutive activation of this pathway produced by aberrant receptor kinases, such as BCR/Abl and Her2/Neu, leads to persistent phosphorylation and activation of Akt kinase. Activated Akt can then phosphorylate pro-apoptotic proteins (shown in green) including Caspase-9 and Bad leading to their inactivation. Activated Akt also activates IκK leading to activation of NFκB transcriptional activity that leads to expression of anti-apoptotic proteins A1, Bcl-xL, and inducible Nitric Oxide Synthase (shown in red). Activation of PP2A, for example by treatment with ApoE peptides, reverses the constitutive activation of this signaling pathway by directly dephosphorylating Akt, IκK, and Bad. (B) TCL1-Akt signaling pathway in B-cell chronic lymphocytic leukemia (CLL). Growth and survival factors through their receptors activate PI-3 kinase. PI-3K phosphorylates phospholipids located at the plasma membrane inducing the translocation of Akt kinase to the membrane where it becomes phosphorylated at Thr308 and Ser473 thereby activating the kinase. TCL1, which is overexpressed in mature B cells in CLL, binds Akt kinase further increasing its kinase activity. TCL1 overexpression (as in CLL) increases phosphorylation levels of Akt targets resulting in the resistance to apoptosis and increase in cell survival.

FIG. 2. COG peptides inhibit activation of Akt/NFκB signaling cascade. (A) BV2 microglial cells in 6 well plates were treated with 100 ng/mL of LPS in the presence of 5 μM COG133. Cells were harvested, lysed in Laemmli sample buffer, run on 10% polyacrylamide gels and Western blotted to nitrocellulose. Blots were probed with an anti-phospho-IκBα antibody or the cognate anti-IκBα antibody. (B) NFκB nuclear translocation was monitored by lysing isolated nuclei from stimulated BV2 microglial cells and incubating with a 32P end-labeled κB binding oligo nucleotide and run on a polyacrylamide gel. The gel was dried and exposed to X-ray film. The arrows indicate the position of NFκB. (C) Densitometry analysis of Western blots probed for phospho-Akt kinase and actin isolated from microglia stimulated with LPS alone or in the presence of COG112 peptide. The signal from phosphorylated Akt kinase was normalized to that of actin. A representative blot probed for phospho-Akt kinase is shown below the bar graph. (D) YAMC cells were exposed to C. rodentium in the presence (closed squares) or absence (open squares) of COG112 peptide. IκK activity was measured by ELISA assay in cellular lysates of the stimulated cells at the times indicated. Time 0 indicates cells that were not stimulated with C. rodentium. §p<0.05, §§p<0.01 versus C. rodentium alone; n=3 separate experiments performed in duplicate.

FIG. 3. Activation of PP2A by treatment with COG112. RAW cell cultures were treated with the indicated compounds for 30 minutes followed by lysis in an NP40 lysis buffer. PP2A was immunoprecipitated and assayed for activity.

FIG. 4. Dose response curves for COG112 on CLL or PBMC cells from human patients. The CLL cells were isolated from 7 CLL patients, while the PBMC cells were isolated from 5 healthy patients. Human CLL cells and PBMC were isolated and assayed for cytotoxicity upon exposure of varying concentrations of COG112.

FIG. 5. Dose response curve for COG112 on apoptosis of CLL cells. Human CLL cells were isolated and exposed to increasing concentrations of COG112. Apoptosis was measured by staining the cells with Annexin V-FITC and propidium iodide followed by flow cytometry analysis. The percentage of Annexin-V+/propidium iodide+ cells are plotted versus COG112 concentration. Etoposide was used as a positive control.

FIG. 6. SET is overexpressed in CLL cells. A scatter plot of the SET/β-Actin ratio measured for cell samples from 12 CLL patients (CLL) and 5 normal patients (PBMC) showing a significant increase in expression of SET in B-CLL cells. Representative Western blots for SET and β-actin protein are depicted below the plot.

FIG. 7. Effects of COG112 on BCR/ABL+ K562 CML cells. (A) Dose response curves for COG112 on K562 CML cells. Imatinib is used as a positive control. (B) COG112 and Imatinib exert synergistic effects on K562 CML cell growth. BCR/Abl+ K562 CML cells were grown in the presence of the indicated compounds or combination of compounds and were stained with Trypan blue. The number of trypan blue stained cells were counted and plotted versus time in culture.

FIG. 8. A. Western blot analysis for phospho-BCR/ABL of K562 cells treated with no compound or COG112 at doses of 0.5 or 1.0 μM for 24 hours. B. PP2A activity of K562 cells treated with the indicated doses of COG112 for 24 hours.

FIG. 9. Growth curve of Jurkat T-cell leukemia cells in the absence and presence of 1 μM COG112 peptide.

### DETAILED DESCRIPTION OF THE INVENTION

The present invention is based on the discovery that ApoE peptides can be used to treat various forms of cancer. Accordingly, the present invention provides methods of treating cancer in a subject in need thereof by administering at least one ApoE peptide to the subject.

ApoE peptides, also referred to as COG peptides, are peptides derived from the native ApoE holoprotein. In one embodiment of the invention, the ApoE peptide may comprise residues 133-149 of ApoE. In another embodiment, the ApoE peptide is COG133 (LRVRLASHLRKLRKRLL (SEQ ID NO: 1)). COG133 has previously proven useful in treating or reducing cerebral ischemia or cerebral inflammation. See U.S. Application Publication No. 2003/0077641 A1, filed Sep. 23, 2002, incorporated herein by reference in its entirety. A large number of analogs of the ApoE 130-150 peptide were previously created and their activity tested in a cell-based assay for suppression of release of inflammatory cytokines and free radicals and in receptor binding assays. Lynch et al. (2003) J. Biol. Chem., Vol. 278(4): 48529-33 and U.S. Application Publication Serial No. 2003/0077641 A1, filed Sep. 23, 2002; U.S. Pat. No. 7,205,280, issued Apr. 17, 2007; and U.S. application Ser. No. 09/260,430, filed Mar. 1, 1999, the contents of each of which are incorporated herein by reference in their entireties. ApoE peptides useful in the methods of the present invention may be derivatives of a peptide containing ten or more residues from the native ApoE protein, including derivatives having non-natural amino acid substitutions, such as amino isobutyric acid and acetyl lysine, and other modifications that enhance the alpha-helical content of the peptide. For instance, ApoE peptide derivatives that are suitable for use in the methods of the invention include, but are not limited to:

| | |
|---|---|
| LRVRLASH-(NMe)-LRKLRKRLL-NH₂ | (SEQ ID NO: 16) |
| Ac-ASH-Aib-RKLRKRLL-NH₂ | (SEQ ID NO: 17) |
| Ac-AS-Aib-LRKLRKRLL-NH₂ | (SEQ ID NO: 18) |
| Ac-DS-Aib-LRKLRKRLL-NH₂ | (SEQ ID NO: 19) |
| Ac-ASHLRKL-Aib-KRLL-NH₂ | (SEQ ID NO: 20) |
| Ac-DR-Aib-ASHLRKLRKR-Aib-L-NH₂ | (SEQ ID NO: 21) |
| Ac-DS-Aib-LRKLRKR-Aib-L-NH₂ | (SEQ ID NO: 22) |
| Ac-DR-Aib-ASHLRKL-Aib-KRLL-NH₂ | (SEQ ID NO: 23) |
| Ac-DS-Aib-LRKL-Aib-KRLL-NH₂ | (SEQ ID NO: 24) |
| Ac-DR-Aib-AS-Aib-LRKLRKRLL-NH₂ | (SEQ ID NO: 25) |
| Ac-DR-Aib-ASHLRKLRKRLL-NH₂ | (SEQ ID NO: 26) |
| Ac-CAS-Aib-LRKL-Aib-KRLL-NH₂ | (SEQ ID NO: 27) |
| Ac-DS-Aib-LRKL-Aib-KRLL-NH₂ | (SEQ ID NO: 28) |
| Ac-AS-Aib-LRKL-Aib-KRLV-NH₂ | (SEQ ID NO: 29) |
| Ac-AS-Aib-LRKL-Aib-KRLM-NH₂ | (SEQ ID NO: 30) |
| Ac-AS-Aib-LRKL-Aib-KRLI-NH₂ | (SEQ ID NO: 31) |
| Ac-AS-Aib-LRKL-Aib-KRLA-NH₂ | (SEQ ID NO: 32) |
| Ac-AS-Aib-LRKL-Aib-KALL-NH₂ | (SEQ ID NO: 33) |
| Ac-AS-Aib-LRKL-Aib-K(orn)LL-NH₂ | (SEQ ID NO: 34) |
| Ac-AS-Aib-LRKL-Aib-K(narg)LL-NH₂ | (SEQ ID NO: 35) |
| Ac-AS-Aib-LRKL-Aib-K(harg)LL-NH₂ | (SEQ ID NO: 36) |
| Ac-AS-Aib-LRKL-Aib-K(dmarg)LL-NH₂ | (SEQ ID NO: 37) |
| Ac-AS-Aib-LRKL-Aib-ARLL-NH₂ | (SEQ ID NO: 38) |
| Ac-AS-Aib-LRKL-Aib-(aclys)RLL-NH₂ | (SEQ ID NO: 39) |
| Ac-AS-Aib-LRKL-Aib-(azlys)RLL-NH₂ | (SEQ ID NO: 40) |
| Ac-ASH-Aib-RKL-Aib-KRLL-NH₂ | (SEQ ID NO: 41) |
| Ac-AS-Aib-LRKL-Aib-KRL-(NLe)-NH₂ | (SEQ ID NO: 42) |
| Ac-AS-Aib-LRKL-Aib-KR-(NLe)-L-NH₂ | (SEQ ID NO: 43) |
| Ac-AS-Aib-LRKL-Aib-KR-(NLe)- | (SEQ ID NO: 44) |
| (NLe)-NH₂ | |
| Ac-AS-Aib-LRKL-Aib-K(orn)L-(NLe)- | (SEQ ID NO: 45) |
| NH₂ | |
| Ac-AS-Aib-LRKL-Aib-K(orn)-(NLe)- | (SEQ ID NO: 46) |
| L-NH₂ | |
| Ac-AS-Aib-LRKL-Aib-K(orn)-(NLe)- | (SEQ ID NO: 47) |
| (NLe)-NH₂ | |
| Ac-AS-Aib-LRKL-Aib-K(harg)L- | (SEQ ID NO: 48) |
| (NLe)-NH₂ | |
| Ac-AS-Aib-LRKL-Aib-K(harg)-(NLe)- | (SEQ ID NO: 49) |
| L-NH₂ | |
| Ac-AS-Aib-LRKL-Aib-K(harg)-(NLe)- | (SEQ ID NO: 50) |
| (NLe)-NH₂ | |
| Ac-AS-Aib-L(orn)KL-Aib-KRLL-NH₂ | (SEQ ID NO: 51) |
| Ac-AS-Aib-L(orn)KL-Aib-K(orn)LL- | (SEQ ID NO: 52) |
| NH₂ | |
| Ac-AS-Aib-L(orn)KL-Aib-KRL-(NLe)- | (SEQ ID NO: 53) |
| NH₂ | |
| Ac-AS-Aib-L(orn)KL-Aib-KRL-(NLe)- | (SEQ ID NO: 54) |
| (NLe)-NH₂ | |
| Ac-AS-Aib-L(orn)KL-Aib-K(orn)L- | (SEQ ID NO: 55) |
| (NLe)-NH₂ | |
| Ac-AS-Aib-L(orn)KL-Aib-K(orn)- | (SEQ ID NO: 56) |
| (NLe)-(NLe)-NH₂ | |
| Ac-ASHLRKLRKRLL-NH₂ (apoe138-149) | (SEQ ID NO: 57) |
| Ac-ASHCRKLCKRLL-NH₂ | (SEQ ID NO: 58) |
| Ac-ASCLRKLCKRLL-NH₂ | (SEQ ID NO: 59) |
| Ac-CSHLRKLCKRLL-NH₂ | (SEQ ID NO: 60) |
| Ac-ASHLRKCRKRCL-NH₂ | (SEQ ID NO: 61) |
| Ac-ASHCRKLRKRCL-NH₂ | (SEQ ID NO: 62) |

wherein (NMe)-L is an N-methylated Leucine, Aib is amino iso-butyric acid, (orn) is ornithine, (narg) is nitroarginine, (NLe) is neurleucine, (harg) is homoarginine, (dmarg) is dimethyl arginine, (aclys) is acetyl lysine, (azlys) is azalysine and Ac is an acelyated carboxy terminus.

In some embodiments, the ApoE peptides may bind to the endogenous inhibitor-2 of protein phosphatase 2A (I₂^{PP2A}) also known as SET as described in WO 2008/080082, which is herein incorporated by reference in its entirety. In other embodiments, the ApoE peptides are analogs or derivatives of COG133, a peptide having the sequence LRVRLASHLRKLRKRLL (SEQ ID NO: 1). In another embodiment, the ApoE peptide is COG1410 (Ac-AS-Aib-LRKL-Aib-KRLL-NH2 (SEQ ID NO: 4)). In another embodiment, the ApoE peptide is COG345 (LRVRLAS-aib-LRKLRK(ac)RLL (SEQ ID NO: 6)).

In another embodiment of the invention, the efficacy of COG133 and other ApoE peptides can be improved by conjugation to a protein transduction domain (PTD) as described in PCT application WO 2006/029028, filed Sep. 2, 2005, which claims priority to U.S. Provisional Applications 60/606,506, filed Sep. 2, 2004, 60/608,148, filed Sep. 9, 2004, 60/606,507, filed Sep. 2, 2004, which are herein incorporated by reference in their entireties. PTDs are short basic peptides that promote the intracellular delivery of cargo that would otherwise fail to, or only minimally, traverse the cell membrane. Some non-limiting examples of PTDs that may be conjugated to the ApoE peptides include antennapedia, SynB 1, SynB3, SynB5, TAT, and polyarginine. For instance, exemplary PTD sequences that can be conjugated to the ApoE peptides of the invention include:

| | |
|---|---|
| GRKKRRQRRRPPQ | (SEQ ID NO: 9) |
| RQIKIWFQNRRMKWKK | (SEQ ID NO: 10) |
| RRMKWKK | (SEQ ID NO: 11) |
| RGGRLSYSRRRFSTSTGR | (SEQ ID NO: 12) |
| RRLSYSRRRF | (SEQ ID NO: 13) |
| RGGRLAYLRRRWAVLGR | (SEQ ID NO: 14) |
| RRRRRRRR | (SEQ ID NO: 15) |

Other suitable carriers are disclosed in U.S. Pat. No. 7,205,280, which is herein incorporated by reference in its entirety. In a preferred embodiment, the ApoE peptide is conjugated to antennapedia. In another preferred embodiment, the ApoE peptide is COG112 (RQIKIWFQNRRMKWKKCLRVRLASHLRKLRKRLL (SEQ ID NO: 2)). In one embodiment, the ApoE peptide is conjugated to SynB3. In another embodiment, the ApoE peptide is COG068 (RRLSYSRRRFLRVRLASHLRKLRKRLL (SEQ ID NO: 8)).

In addition, agents such as COG1410 are of enhanced efficacy, and demonstrate a greater therapeutic index. As used herein, “therapeutic index” refers to the maximum tolerated dose at which no animal dies divided by the minimal effective dose at which performance after injury is significantly better than saline controls.

Without being bound to any theory, the inventors of the present invention have reason to believe that COG133 and derivatives thereof are activators of protein phosphatase 2A (PP2A). Accordingly, in one embodiment of the invention, ApoE peptides increase the activity of PP2A in treated cells. Activation of PP2A by ApoE peptides may also decrease activity of Akt kinase, IκK kinase, and NFκB, thereby promoting induction of apoptosis. Thus, in some embodiments, ApoE peptides decrease Akt kinase activity in treated cells. In other embodiments, ApoE peptides induce apoptosis of treated cells, i.e. cancer cells.

Peptides of the present invention can be produced by standard techniques as are known in the art. The peptides of the invention may have attached various label moieties such as radioactive labels, heavy atom labels and fluorescent labels for detection and tracing. Fluorescent labels include, but are not limited to, luciferin, fluorescein, eosin, Alexa Fluor, Oregon Green, rhodamine Green, tetramethylrhodamine, rhodamine Red, Texas Red, coumarin and NBD fluorophores, the QSY 7, dabcyl and dabsyl chromophores, BODIPY, Cy5, etc.

Modification of the peptides disclosed herein to enhance the functional activities associated with these peptides could be readily accomplished by those of skill in the art. For instance, the peptides used in the methods of the present invention can be chemically modified or conjugated to other molecules in order to enhance parameters such as solubility, serum stability, etc., while retaining functional activity. In particular, the peptides of the invention may be acetylated at the N-terminus and/or amidated at the C-terminus, or conjugated, complexed or fused to molecules that enhance serum stability, including but not limited to albumin, immunoglobulins and fragments thereof, transferrin, lipoproteins, liposomes, α-2-macroglobulin and α-1-glycoprotein, PEG and dextran. Such molecules are described in detail in U.S. Pat. No. 6,762,169, which is herein incorporated by reference in its entirety.

Another variation of the peptide agents of the present invention is the linking of from one to fifteen amino acids or analogs to the N-terminal or C-terminal amino acid of the therapeutic peptide. Analogs of the peptides of the present invention can also be prepared by adding from one to fifteen additional amino acids to the N-terminal, C-terminal, or both N- and C-terminals, of an active peptide, where such amino acid additions do not adversely affect the ability of the peptide to bind to receptors at the site bound by peptides of the invention. For instance COG133, COG1410, and COG345 variants can be created by adding from one to fifteen additional amino acids to the N-terminal, C-terminal, or both N- and C-terminals, of the active peptide.

The ApoE peptides of the present invention further include conservative variants of the peptides herein described. As used herein, a conservative variant refers to alterations in the amino acid sequence that do not adversely affect the biological functions of the peptide. A substitution, insertion or deletion is said to adversely affect the peptide when the altered sequence prevents or disrupts a biological function associated with the peptide. For example, the overall charge, structure or hydrophobic/hydrophilic properties of the peptide may be altered without adversely affecting a biological activity. Accordingly, the amino acid sequence can be altered, for example to render the peptide more hydrophobic or hydrophilic, without adversely affecting the biological activities of the peptide. Ordinarily, the conservative substitution variants, analogs, and derivatives of the peptides, will have an amino acid sequence identity to the disclosed sequences SEQ ID NOs: 1, 2, 4, and 6 of at least about 55%, at least about 65%, at least about 75%, at least about 80%, at least about 85%, at least about 90%, at least about 95%, or at least about 96% to 99%. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N-terminal, C-terminal or internal extensions, deletions, or insertions into the peptide sequence shall not be construed as affecting homology.

Thus, the peptides of the present invention include molecules having the amino acid sequence disclosed in SEQ ID NOs: 1, 2, 4, or 6; fragments thereof having a consecutive sequence of at least about 3, 4, 5, 6, 10, 15, or more amino acid residues of the therapeutic peptide; amino acid sequence variants of such peptides wherein an amino acid residue has been inserted N- or C-terminal to, or within, the disclosed sequence; and amino acid sequence variants of the disclosed sequence, or their fragments as defined above, that have been substituted by another residue. Peptide compounds comprising the peptide sequences of the invention may be between about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids or more. Contemplated variants further include those containing predetermined mutations by, e.g., homologous recombination, site-directed or PCR mutagenesis, and the corresponding peptides of other animal species, including but not limited to rabbit, rat, porcine, bovine, ovine, equine and non-human primate species, and derivatives wherein the peptide has been covalently modified by substitution, chemical, enzymatic, or other appropriate means with a moiety other than a naturally occurring amino acid (for example, a detectable moiety such as an enzyme or radioisotope).

The ApoE peptides, including but not limited to COG133, COG112, and derivatives thereof, can be in free form or the form of a salt, where the salt is pharmaceutically acceptable. These include inorganic salts of sodium, potassium, lithium, ammonium, calcium, magnesium, iron, zinc, copper, manganese, and the like. Various organic salts of the peptide may also be made with, including, but not limited to, acetic acid, propionic acid, pyruvic acid, maleic acid, succinic acid, tartaric acid, citric acid, benozic acid, cinnamic acid, salicylic acid, etc.

In one embodiment, the peptides of the present invention are used in combination with a pharmaceutically acceptable carrier. The present invention thus also provides pharmaceutical compositions suitable for administration to a subject. Such compositions comprise an effective amount of the ApoE peptide of the present invention in combination with a pharmaceutically acceptable carrier. The carrier can be a liquid, so that the composition is adapted for parenteral administration, or can be solid, i.e., a tablet or pill formulated for oral administration. Further, the carrier can be in the form of a nebulizable liquid or solid so that the composition is adapted for inhalation. When administered parenterally, the composition should be pyrogen free and in an acceptable parenteral carrier. Active agents can alternatively be formulated encapsulated in liposomes, using known methods. Preparation of a peptide of the present invention for intranasal administration can be carried out using techniques as are known in the art. The inventive peptides may also be formulated for topical administration, for example in the form of creams or gels. Topical formulations are particularly useful for treating skin cancers. In other embodiments, the ApoE peptides may be formulated for rectal administration, such as in the form of suppositories. In some embodiments, rectal administration of the ApoE peptides may be preferred for treatment of colorectal cancer.

Pharmaceutical preparations of the peptides of the present invention can optionally include a pharmaceutically acceptable diluent or excipient.

An effective amount of the ApoE peptide of the present invention is that amount that decreases at least one symptom or pathology associated with cancer, such as tumor size, tumor growth, spread of cancer cells, number of cancer cells, and survival, compared to that which would occur in the absence of the peptide. In one embodiment, the effective amount of an ApoE peptide modulates Akt kinase activity or PP2A activity in a cell in a subject. The effective amount (and the manner of administration) will be determined on an individual basis and will be based on the specific peptide being used and a consideration of the subject (size, age, general health), the specific cancer being treated (e.g. CLL, CML, breast cancer), the severity of the symptoms to be treated, the result sought, the specific carrier or pharmaceutical formulation being used, the route of administration, and other factors as would be apparent to those skilled in the art. The effective amount can be determined by one of ordinary skill in the art using techniques as are known in the art. Therapeutically effective amounts of the peptides described herein can be determined using in vitro tests, animal models or other dose-response studies, as are known in the art.

An alternative method of administering peptides of the present invention is carried out by administering to the subject a vector carrying a nucleic acid sequence encoding the peptide, where the vector is capable of entering cells of the body so that the peptide is expressed and secreted. Suitable vectors are typically viral vectors, including DNA viruses, RNA viruses, and retroviruses. Techniques for utilizing vector delivery systems and carrying out gene therapy are known in the art. Herpesvirus vectors, adenovirus vectors, adeno-associated virus vectors and lentiviral vectors are particular types of vectors that can be employed in administering compounds of the present invention.

The peptides of the present invention may be used alone to treat cancer or in combination with other therapeutic agents commonly used to treat cancer, such as, e.g. chemotherapy agents (chlorambucil, cyclophosphamide), corticosteroids (prednisone, prednisolone), fludarabine, pentostatin, cladribine, imatinib (Gleevec), dasatinib (Sprycel), hormonal therapy (tamoxifen, aromatase inhibitors), and radiation.

The peptides of the present invention can be administered acutely (i.e., during the onset or shortly after events leading to a cancer diagnosis), or can be administered prophylactically (e.g., before scheduled surgery, or before the appearance of cancer signs or symptoms), or administered during the course of a cancer to reduce or ameliorate the progression of symptoms that would otherwise occur. The timing and interval of administration is varied according to the subject's symptoms, and can be administered at an interval of several hours to several days, over a time course of hours, days, weeks or longer, as would be determined by one skilled in the art.

The typical daily regime can be from about 0.01 μg/kg body weight per day, from about 1 mg/kg body weight per day, from about 10 mg/kg body weight per day, from about 100 mg/kg body weight per day, from about 1,000 mg/kg body weight per day. Depending on the particular ApoE peptide to be administered, dosages can be between about 1 mg/kg and about 500 mg/kg body weight per day, preferably between about 25 mg/kg and about 400 mg/kg body weight per day, or more preferably between about 50 mg/kg and about 250 mg/kg body weight per day.

The present invention provides methods of treating cancer in a mammalian subject in need thereof by administering an effective amount at least one ApoE peptide as described herein. ApoE peptides can reduce one or more symptoms associated with cancer, including but not limited to tumor formation, tumor growth, number of cancerous cells, spread of cancerous cells to healthy tissue, and decreased survival. Cancers that may be treated with the peptides and methods of the invention include, but are not limited to, various forms of leukemia (CLL, CML, ALL, AML), breast cancer, ovarian cancer, cervical cancer, prostate cancer, colorectal cancer, lung cancer, pancreatic cancer, brain cancer, skin cancer (melanoma and nonmelanoma), head and neck cancers, bladder cancer, endometrial cancer, renal cell cancer, thyroid cancer, stomach cancer, esophageal cancer, gall bladder cancer, liver cancer, lymphoma, and sarcoma. In one embodiment, the ApoE peptides can reduce activation of signaling pathways, such as the Akt/NFκB pathway, that are aberrantly activated in various forms of cancer (see Example 1). ApoE peptides can also activate PP2A (Example 2). PP2A has been reported to negatively regulate endothelial cell motility, which is required for angiogenesis and tumor metastasis in cancers (Gabel et al., 1999, Otolaryngol Head Neck Surg. 121: 463-468; Young, M R., 1997, Adv Exp Med Biol. 407: 311-318) Inhibition of PP2A by okadaic acid increased cell motility by disrupting the cytoskeletal network thereby enhancing the invasive properties of the tumor cells. Thus, peptides of the present invention would reduce tumor cell metastasis and cancer-associated angiogenesis by activating PP2A. In one embodiment of the invention, administration of the ApoE peptide increases PP2A activity in a cancer cell of the subject. In another embodiment, administration of the ApoE peptide decreases Akt kinase activity in a cancer cell of the subject. In another embodiment, administration of the ApoE peptide decreases IκK kinase activity in a cancer cell of the subject. In another embodiment, administration of the ApoE peptide decreases NFκB kinase activity in a cancer cell of the subject. In still another embodiment, administration of the ApoE peptide induces apoptosis of a cancer cell in the subject.

The present invention also provides a method for the treatment of leukemia comprising administering at least one ApoE peptide in an amount that would reduce symptoms of the disease as compared to that which would occur in the absence of the peptide. In one embodiment, the leukemia is chronic myelogenous leukemia (CML). SET, an endogenous negative regulator of PP2A, is overexpressed in CML and inhibits PP2A, thus maintaining activation of the oncogenic BCR/ABL kinase pathway (Neviani et al. (2005) Cancer Cell. 8: 355-368). Therefore, administration of an ApoE peptide, such as COG133, COG1410, COG112 or any other ApoE analog, would activate PP2A, which would then be free to dephosphorylate regulators of cell proliferation and survival as well as suppress the oncogenic activity of the BCR/ABL kinase thus reducing leukemogenesis. In some embodiments, administration of the ApoE peptide decreases the growth of BCR/ABL+ cells in the subject. In certain embodiments, BCR/ABL+ cells are resistant to imatinib (Gleevec) and/or dasatinib (Sprycel), that is the growth of such imatinib- and/or dasatinib-resistant cells is not inhibited by either of these compounds. Without being bound by theory, it is believed that ApoE peptides can effectively inhibit the growth of imatinib- or dasatinib-resistant cells by increasing PP2A activity within the cells, which in turn dephosphorylates and deactivates BCR/ABL kinase. Such a mechanism is distinct from that of imatinib or dasatinib, which act on the BCR/ABL kinase directly and are affected by mutations of the kinase. In another embodiment, the leukemia is chronic lymphocytic leukemia (CLL). In preferred embodiments, administration of the ApoE peptide decreases the number of CD5+ B cells in the subject. In another embodiment, the leukemia is acute lymphocytic leukemia (ALL).

The present invention also encompasses methods of treating breast cancer in a subject by administering an effective amount of at least one ApoE peptide to the subject. In one embodiment, the breast cancer is characterized by Her2 expression. In another embodiment, the breast cancer is characterized by estrogen receptor expression. Administration of ApoE peptides preferably reduce tumor growth following their administration.

In certain embodiments, the invention provides pharmaceutical compositions comprising at least one ApoE peptide. In certain embodiments, the invention provides pharmaceutical compositions comprising at least one ApoE peptide with another drug for the treatment, prevention or amelioration of cancer. The pharmaceutical compositions of the peptides of the present invention can be provided in such a way as to facilitate administration to a subject in need thereof, including, for example, by intravenous, intramuscular, subcutaneous or transdermal administration. See, Remington's Pharmaceutical Sciences, 19th ed. Remington and Gennaro, eds. Mack Publishing Co., Easton, Pa., incorporated herein by reference. The methods of the present invention further provide for various dosing schedules, administration times, intervals and duration to treat, prevent or ameliorate cancer, such as CLL, CML, and breast cancer. Also included are functional variants of the disclosed peptides as known in the art. Consistent therewith, the invention also includes use of the disclosed peptides and functional variants thereof in methods of making medicaments for treating various forms of cancer as discussed herein.

The examples which follow are set forth to illustrate the present invention, and are not to be construed as limiting thereof.

### EXAMPLES

### Example 1

### COG Peptides Modulate the Akt/NFκB Pathway

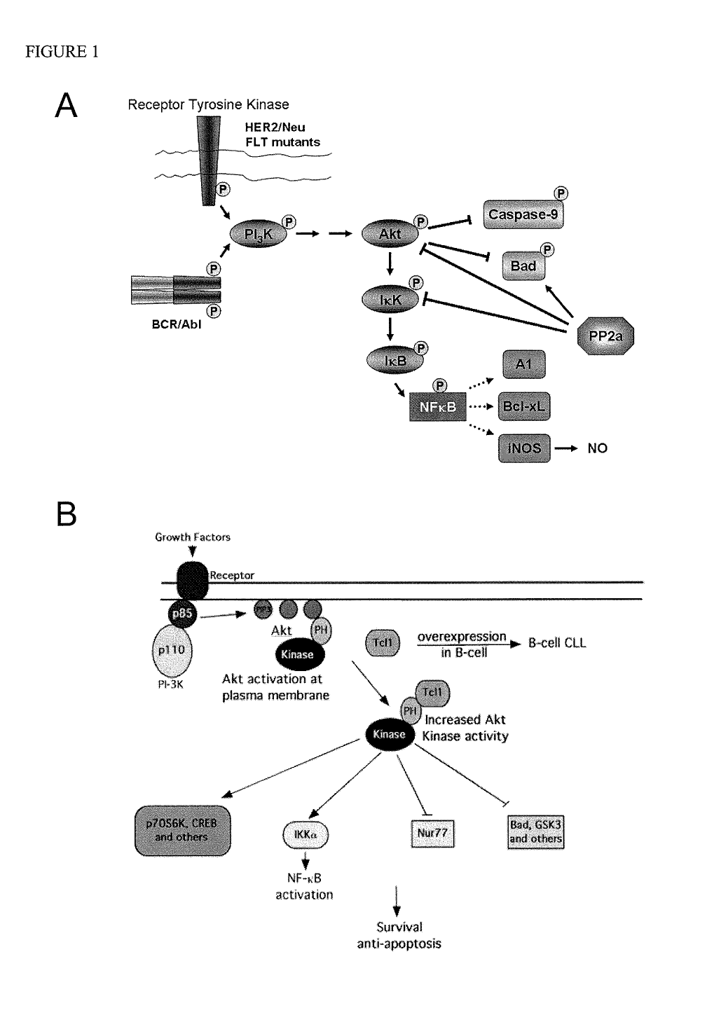

Many types of cancer feature aberrant, constitutive activation of the phosphatidylinositol-3 Kinase (PI-3K)/Akt pathway, which results in establishment of an anti-apoptotic environment in the cancer cell that correlates with poor outcome. When growth factors such as insulin activate the PI3 Kinase at the plasma membrane, phosphoinositides are phosphorylated leading to the translocation of Akt to the plasma membrane where it is activated by phosphorylation at Thr308 and Ser473. Once activated, Akt regulates proteins that are essential for cell survival through two mechanisms (FIG. 1). First, Akt can regulate survival proteins by controlling the function of these survival proteins through kinase-mediated activation or inhibition. It has been demonstrated that activated Akt directly phosphorylates caspase-9 and Bad thereby inactivating them. Caspase-9 is a protease that is activated early in the normal apoptosis cascade, while Bad is a pro-apoptotic protein of the Bcl-2 family that binds to and inhibits the pro-survival function of Bcl-xL. Phosphorylation of Bad by Akt inhibits its pro-apoptotic activity and increases the pro-growth cancerous state in the cell. The second mechanism by which activated Akt shifts a cell to an anti-apoptotic state is through transduction of signals that increase transcription and production of survival proteins. For example, Akt activation increases the expression of Mcl-1 by activating the IKB Kinase (IκK), which in turn phosphorylates IκB, the endogenous inhibitor of NFκB, leading to the release and activation of NFκB. Other NFκB-regulated anti-apoptotic genes include Bcl-xL and A1 as well as inducible nitric oxide synthase (iNOS). Upregulation of iNOS has independently been shown to correlate with rapid progression, frequency of relapse, and death rate in breast cancer patients.

In approximately 30% of breast cancers, constitutive Akt activation can be traced to enhanced expression of the HER2/Neu gene product, which is a constitutively activated receptor tyrosine kinase that activates the PI-3 kinase leading to Akt activation. A similar activation of PI-3K is responsible for the induction of chronic myelogeneous leukemia by the BCR/Abl fusion protein (FIG. 1A). The T cell leukemia/lymphoma 1 oncogene (TCL1), which has been implicated in the pathogenesis of B-cell chronic lymphocytic leukemia (B-CLL), was found to directly bind Akt and enhance Akt kinase activity (Pekarsky et al. (2000) Proc. Natl. Acad. Sci. USA, Vol. 97: 3028-3033). Thus, TCL1 expression in mature B-cells would be expected to produce enhanced Akt kinase activity and increase the production of multiple anti-apoptotic factors leading to the disrupted apoptosis characteristic of CLL (FIG. 1B).

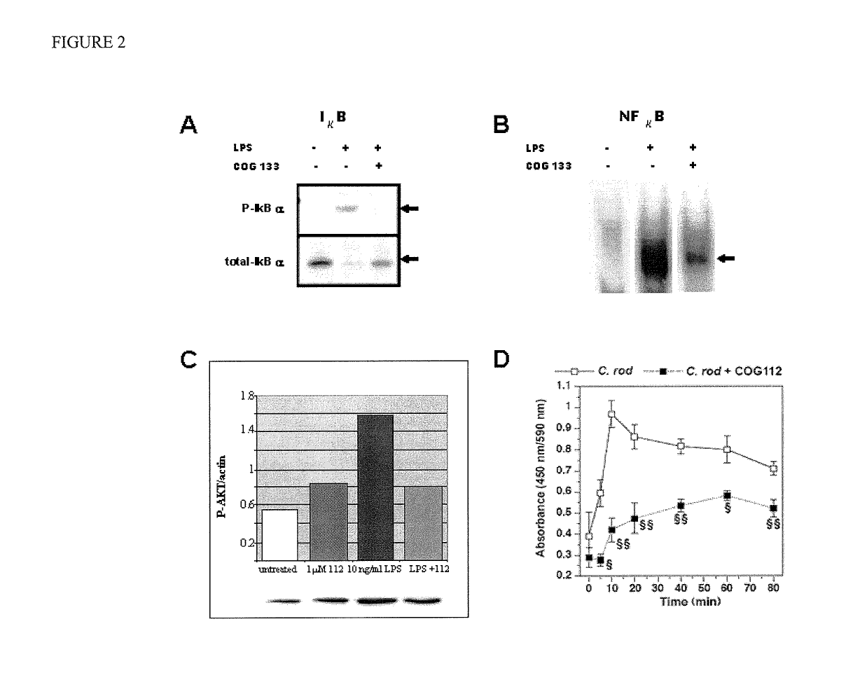

To determine whether COG peptides modulated the PI-3 kinase/Akt pathway and subsequent activation of NFκB, the phosphorylation state of proteins in the pathway was analyzed in cells exposed to bacteria or a bacterial antigen (lipopolysaccharide) in the absence and presence of COG peptides. In a first series of experiments, mouse BV2 microglia in six well plates were incubated with 100 ng/mL of LPS alone or in the presence of 5 μM COG133 (SEQ ID NO: 1) peptide. Cells were lysed and a clarified extract prepared by centrifugation. Polyacrylamide gels were loaded with 30 μg of extract per lane and run in SDS buffer. Proteins were transferred from the gel onto nitrocellulose membranes, which were subsequently blocked with 10% nonfat dry milk. Membranes were then probed with an anit-phospho-IκBα antibody. Membranes were developed with Enhanced Chemiluminescence substrates (GE healthcare) and visualized by exposure to film. Membranes were then stripped and reprobed for total IκB using a non phospho-specific anti-IκBα antibody. A duplicate blot was probed with anti-GAPDH antibodies. As shown in FIG. 2A, the COG133 peptide reduces the LPS-induced phosphorylation of IKB.

In the dephosphorylated state, IκB binds to the transcription factor NFκB and prevents it from translocating to the nucleus to activate transcription of pro-survival proteins. To determine if COG133 also reduced the translocation of NFκB to the nucleus, BV2 microglial cells were stimulated with LPS alone or in the presence of COG133 and nuclear extracts were prepared from the stimulated cells. A radiolabeled oligonucelotide containing a NFκB binding site was added to the proteins from the nuclear extracts and the proteins were subsequently separated by non-denaturing polyacrylamide gel electrophoresis. The amount of NFκB present in the nuclear extracts was detected by autoradiography (FIG. 2B). Nuclear NFκB was reduced in the presence of COG133 providing further evidence that this signal transduction cascade is suppressed in cells treated with ApoE peptides.

To determine if COG peptides could also suppress activation of the upstream Akt kinase, BV2 microglial cells were treated with 10 ng/mL LPS alone or in the presence of 1 μM COG112 (SEQ ID NO: 2) peptide. Cell lysates were run on 10% polyacrylamide gels and subsequently transferred to nitrocellulose membranes. Blots were probed with an anti-phospho-Akt antibody or an antibody to actin. Densitometry analysis of the western blots was conducted and the signals from phosphorylated Akt kinase were normalized to those from actin. The results of the densitometry analysis are shown in FIG. 2C. COG112 significantly decreased LPS-induced phosphorylation of the Akt kinase.

Akt kinase can phosphorylate and activate IκB Kinase (IκK), which leads to the de-repression of NFκB by IκB. As described above, COG peptides reduce phosphorylation of IκB and subsequent activation of NFκB induced by a Toll-like receptor 4 agonist (LPS). To determine if COG peptides also affect IκK activation, IκK activity was assessed in cytoplasmic cellular lysates of young adult mouse colon (YAMC) cells exposed to C. rodentium bacteria alone or in the presence of COG112 (SEQ ID NO: 2). IκK activity was measured using a specific ELISA assay (K-LISATM detection kit, Calbiochem/EMD Biosciences). Cytosolic cell extracts were incubated in glutathionine coated wells of a 96-well plate with a glutathione S transferase (GST)-tagged IκB-α fusion polypeptide substrate that includes the Ser32 and Ser36 IκB-α kinase phosphorylation sites. Phosphorylated GST-IκB-α substrate was detected using horseradish peroxidase-conjugated anti-phospho-IκB-α antibody. Optical density proportional to kinase activity was measured at 450 nm in a plate reader. The time course of IκK activation induced by C. rodentium in the presence (closed squares) or absence (open squares) of COG112 is shown in FIG. 2D. Time 0 indicates cells that were not stimulated with C. rodentium. Similar to the suppression of IκB phosphorylation and NFκB activation observed with COG133 treatment, COG112 substantially reduced IκK activity.

The results of these experiments show that COG peptides are able to reduce activation of the Akt kinase as well as downstream signaling induced by a stimulus, suggesting that COG peptides would be able to effectively modulate overactivation of the Akt/NFκB signaling pathway.

### Example 2

### COG112 activates PP2A

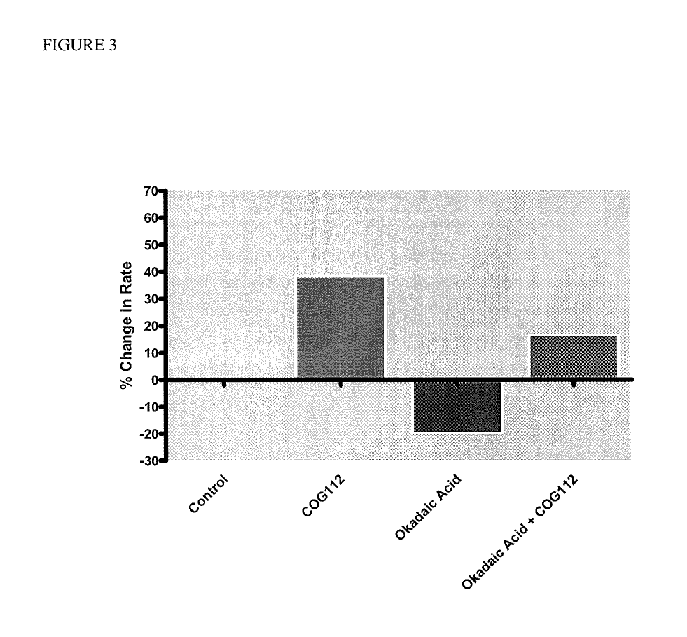

Mouse macrophagic RAW cells were incubated with either 2 μM COG112 (SEQ ID NO: 2), 10 nM okadaic acid (an inhibitor of PP2A), or okadaic acid and COG112. After 30 minutes, cells were lysed and PP2A was immunoprecipitated by adding an antibody targeted to the catalytic C-subunit of PP2A. Half of the immunoprecipitate was separated by SDS-PAGE, blotted on to nitrocellulose, and probed with an anti-PP2AC antibody. The remaining portion was assayed for activity by adding 125 μL assay cocktail containing a phospho-threonine substrate peptide to the immunoprecipitated enzyme. After incubating at 37° C. with shaking, a 25 μL aliquot was removed and added to an ammonium molybdate solution (Upstate) that chelates free phosphate and changes color upon chelate formation. Aliquots were removed at various time intervals and the amount of free phosphate released from the peptide was determined by comparison to a phosphate standard curve. The rate of phosphate release was determined by linear fit to the time course data and was normalized to the relative PP2A concentration. PP2A activity, measured by the rate of phosphate release, was increased in the presence of COG112 (FIG. 3), suggesting that the ApoE peptide relieves a baseline inhibition of PP2A activity. PP2A activity was reduced in the presence of okadaic acid alone as expected. However, COG112 increased PP2A activity in the presence of okadaic acid suggesting that an equilibrium exists between active PP2A and inactive PP2A in the cell and this equilibrium can be shifted by COG112 to modulate the amount of active PP2A enzyme (FIG. 3). Thus, the active pool of PP2A in a cell can be regulated by ApoE peptides.

### Example 3

### COG112 Kills B-CLL Cells in vitro

The persistent anti-apoptotic state of B-cells in chronic lymphocytic leukemia (B-CLL) is thought to be due, in part, to an overactivation of the Akt/NFκB signaling cascade. PP2A is known to counterbalance this signaling pathway by dephosphorylating and inactivating Akt kinase and IκK kinase (Kuo et al. (2008) J Biol Chem., Vol. 283: 1882-1892; Kray et al. (2005) J Biol Chem., Vol. 280: 35974-82). PP2A can also regulate apoptosis pathways by dephosphorylating and activating caspases, which play an early role in the induction of apoptosis. Thus, compromised PP2A activity in a cell would contribute to the constitutive activation of the Akt pathway preserving the anti-apoptotic state. In fact, a deletion at 11g22-q23, which includes a portion of the PPP2R1B gene, represents the second most common chromosomal aberration in B-CLL. The PPP2R1B gene encodes the Aβ constant regulatory subunit of PP2A, commonly known as a tumor suppressor. This deletion results in reduced expression of PPP2R1B and is associated with decreased PP2A activity in B-CLL cells (Kalla et al. (2007) Eur J Cancer, Vol. 43: 1328-35). These patients are characterized by poor survival and the CLL tumor cells show increased survival rates.

As shown in Examples 1 and 2, COG peptides can reduce activation of the Akt/NFkB signaling cascade as well as activate PP2A. To determine whether COG peptides could counteract the overactivation of the Akt/NFkB pathway in B-CLL cells and effectively induce apoptosis, cytotoxicity testing was performed on freshly isolated B-CLL cells from CLL patients with COG peptides. Blood from CLL patients was collected and CD5+/CD19+ CLL cells were isolated using the RosetteSep™ Human B Cell Enrichment Cocktail. This method depletes whole blood of T cells, monocytes, and NK cells using a proprietary antibody cocktail containing anti-CD14, anti-CD2 and anti-CD16 antibodies to remove T cells, monocytes, and NK cells, respectively. The antibody cocktail crosslinks unwanted cells in human whole blood to multiple red blood cells forming immunorosettes thereby increasing the density of the rosetted cells, such that they pellet along with the free RBCs when centrifuged over a buoyant density medium such as Ficoll-Paque®. The highly enriched B-cell or B-CLL cells are left at the interface between Ficoll and the plasma.

COG peptides were applied to the isolated B-CLL cells (0.25×10⁶ cells/well in a 96 well plate) for 72 hours, after which viable cells were assessed using the MTS assay (Pharmacia). The concentration of COG peptide that was effective in killing 50% of the input CLL cells (ED50) was determined. As shown in Table I, COG133 (SEQ ID NO: 1) had slightly improved activity relative to the COG056 (SEQ ID NO: 3) control. COG056 is a version of COG133 that contains the same amino acid composition but has a scrambled sequence which eliminates in vitro and in vivo activity of this peptide. COG248 (SEQ ID NO: 5), which is a modified version of COG133, shows enhanced activity compared to COG133. Increased cell penetration provided by the antennapedia homeobox domain protein transduction domain resulted in a robust improvement in the EC50 of COG133 (COG112; SEQ ID NO: 2) to 224±120 nM. Interestingly, COG1410 (SEQ ID NO: 4), which demonstrated improved potency for suppressing NO production relative to COG133 in microglial cells, demonstrated no difference in potency for cytotoxicity against B-CLL cells.

TABLE I
Cytotoxicity of COG compounds on B-CLL cells

| | | EC50 | |
|---|---|---|---|
| Peptide | Sequence | (μM) | Fold Change |
| COG056 | LLRKRLKRLHSALRVRL | 12.9 ± 4.6 | 1.0 |
| (rev133) | (SEQ ID NO: 3) | | |
| COG133 | LRVRLASHLRKLRKRLL | 4.4 ± 1.5 | 2.9 |
| | (SEQ ID NO: 1) | | |
| COG112 | RQIKIWFQNRRMKWKKCLRVRLASHLRKLRKRLL | 0.22 ± 0.13 | 58.6 |
| (Antp-COG133) | (SEQ ID NO: 2) | | |
| COG248 | LRVRLAS(Aib)LKRL(nitroR)KRLL | 2.3 ± 1.3 | 5.5 |
| | (SEQ ID NO: 5) | | |
| | [Aib is amino isobutyric acid and | | |
| | nitroR is a nitroarginine] | | |
| COG1410 | AS(Aib)LRKL(Aib)KRLL | 5.7 ± 3.0 | 2.3 |
| | (SEQ ID NO: 4) | | |
| | [Aib is amino isobutyric acid] | | |

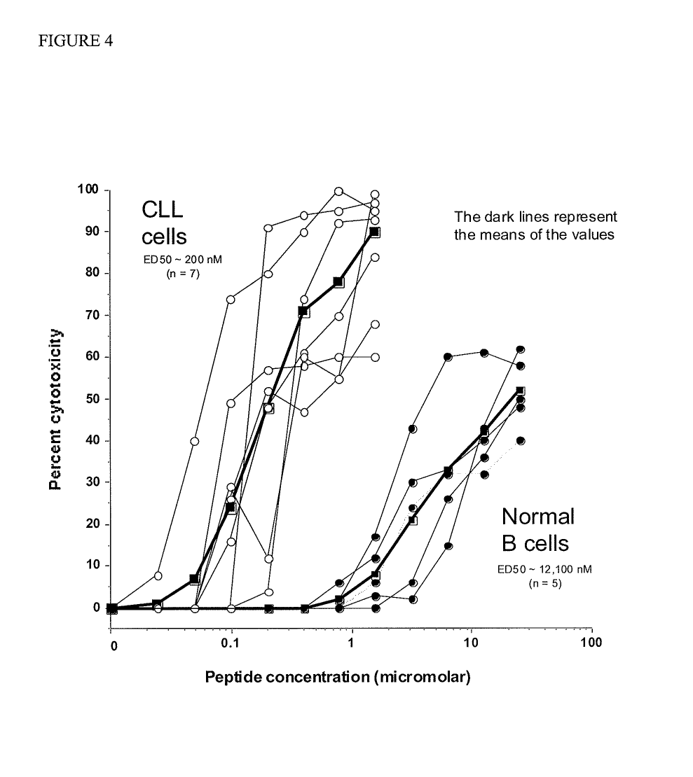

Given the potent cytotoxic activity of COG112 on CLL cells, cytotoxicity testing on normal mononuclear cells was performed to determine if the effect was due to a general cytotoxic effect or if a selective CLL cytotoxic mechanism had occurred. Additional B-CLL cells and peripheral blood mononuclear cells (PBMC; i.e., normal B cells) were isolated from leukemia patients and normal volunteers, respectively, and were treated with a range of COG112 concentrations. As shown in FIG. 4, COG112 was cytotoxic in CLL cells isolated from patients with an EC50 near the 225 nM concentration shown in Table I above. In stark contrast, the EC50 for cytotoxicity in the PBMC from normal volunteers was found to be nearly 2-log units higher at approximately 20 μM. Similar data was obtained from isolated splenic CLL cells from aged TCL-1 transgenic mice (a mouse model for CLL). COG112 was cytotoxic at about 1.5 μM ED50 while the control compound COG056 demonstrated and ED50>25 uM in the transgenic mouse cells. These data demonstrate that ApoE peptides display potent and selective cytotoxic activity for B-CLL cells and provide evidence that ApoE peptides may be a useful therapeutic for B-CLL.

### Example 4

### COG112 Modulates Signal Transduction Cascades in Human B-CLL Cells

As discussed previously, aberrant signaling through the Akt/NFκB pathway is thought to contribute to the persistent anti-apoptotic state of B cells in CLL patients. The downregulation of PP2A in CLL patients contributes to the constitutive activation of the Akt/NFκB pathway and helps to maintain the proliferative state. Here, we have designed experiments to show the effect of COG112 on various signaling cascades within B-cells from human CLL patients and elucidate the mechanism by which COG112 affects these cascades.

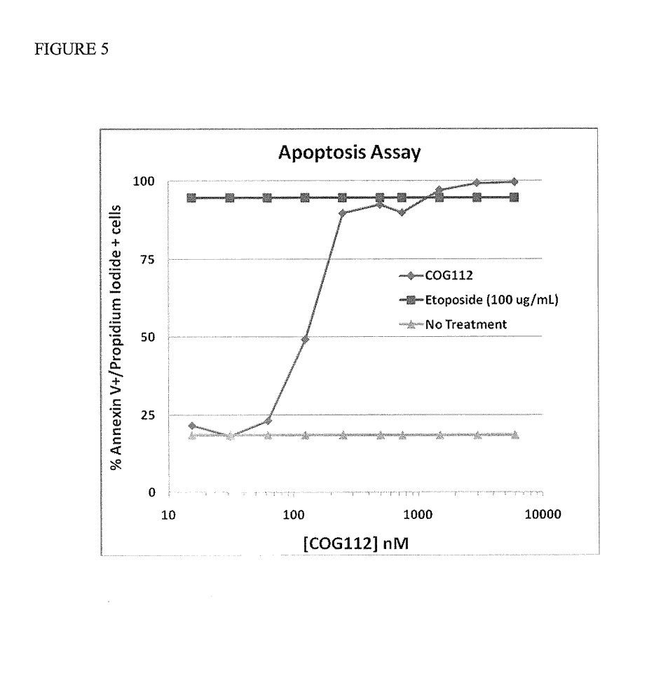

In a first series of experiments, apoptosis assays using Annexin V staining were performed on freshly isolated B-CLL cells to determine if COG112 could reverse the anti-apoptotic state in these cells and induce apoptosis. Blood from CLL patients was collected and CD5+/CD19+ CLL cells were isolated using the RosetteSep™ Human B Cell Enrichment Cocktail (see Example 3) and treated with increasing concentrations of COG112. Apoptosis was measured using the Annexin V-FITC apoptosis detection kit (BD Biosciences-Pharmingen). COG-treated and untreated cells were Annexin V-FITC- and propidium iodide-stained for 15 minutes in 1× binding buffer (10 mM HEPES, pH 7.4, 140 mM NaCl, 2.5 mM CaCl₂) and analyzed by flow cytometry. One aliquot of cells was treated with 100 μg/mL etoposide as a positive control for apoptosis induction. As shown in FIG. 5, a dose-dependent increase in apoptosis was observed with increasing concentrations of COG112. The ED50 for induction of apoptosis closely mirrored the ED50 for cytotoxicity of COG112 on the CLL cancer cells (see Example 3). These results suggest that the cytotoxicity of COG112 on CLL cells occurs at least in part through induction of apoptosis.

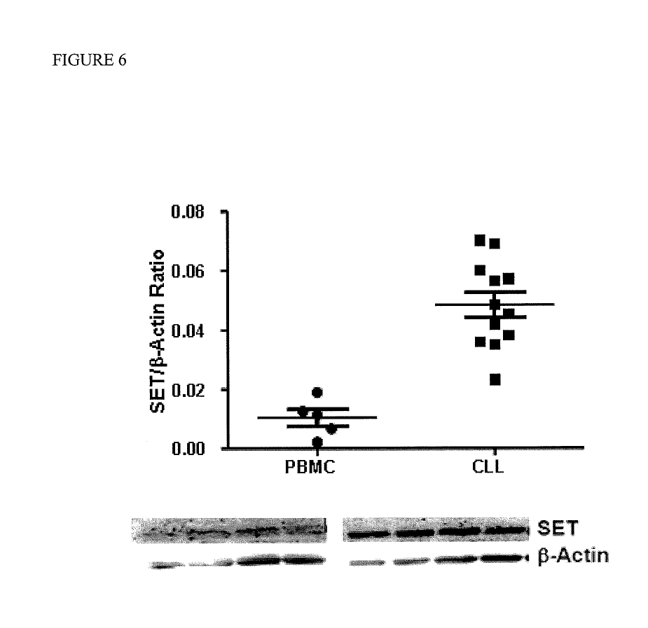

In a second series of experiments, the expression of SET (protein phosphatase 2A inhibitor 2 protein (I₂^{PP2A})) in B-CLL cells was assessed to determine if overexpression of SET contributes to aberrant signaling in CLL as has been reported for CML (Neviani et al. (2006) Cancer Cell, Vol. 8: 355-68). CD19+/CD5+ CLL cells from 12 leukemia patients and CD19+/B-cells from 5 normal volunteers were isolated from whole blood using RosetteSep™ Human B Cell Enrichment Cocktail as described in Example 3. Isolated cells were lysed in an extraction buffer containing phosphate buffered saline with a dissolved Complete protease inhibitor tablet (Boehringer Ingleheim) and phosphatase inhibitors (Roche). For immunoblotting, 40 μg of total protein lysate were loaded for each sample onto a sodium dodecyl sulfate-polyacrylamide gel (SDS-PAGE) and transferred to nitrocellulose membranes. The SET protein was detected using an anti-SET antibody, and was quantitated and normalized using β-Actin as a loading control on a LiCor Odyssey fluorescence scanner. As shown in FIG. 6, there was a statistically significant increase in the SET/β-Actin ratio of CLL samples (0.048±0.004) relative to the SET/β-Actin ratio (0.010±0.003) in the PBMC control group (p<0.0001). This overexpression was independent of apoE genotype or cytogenetic disturbance of the patient. The 4.8 fold overexpression is similar to SET levels reported for diffuse large B-cell lymphoma (Nenasheva et al. (2004) Mol Biol (Mosk), Vol. 38: 265-75; Nenasheva et al. (2005) Int J Med Sci., Vol. 2: 122-128). This overexpression of SET in B-CLL cells would act to decrease PP2A activity and inhibit the ability of PP2A to regulate Akt and the NFκB pathway, thereby promoting the anti-apoptotic state of the cancer cells.

The results of the two previous series of experiments indicate that SET overexpression in CLL cells results in decreased PP2A activity which contributes to the persistence of an anti-apoptotic state in these cells. COG peptides, such as COG112, can induce apoptosis in CLL cells, which may occur through the binding of COG peptides to the SET protein, thereby relieving the suppression of PP2A activity. To further elucidate the mechanism of the cytotoxic effect of COG peptides on CLL cells, the effect of COG112 treatment on the phosphorylation state of key signal transduction proteins and PP2A activity in B-CLL cells is evaluated. Published studies indicate that Erk, Akt, IκK and NFκB are activated in CLL cells leading to increased iNOS expression and NO production. The production of NO is suggested to play a role in the failure of CLL cells to undergo normal apoptosis. B-CLL cells from leukemia patients are isolated using the RosetteSep™ Human B Cell Enrichment Cocktail as described in Example 3. Isolated B cells are plated at 3×10⁶ CLL cells/well and cultured in 24 well tissue culture plates in 1.5 mL of Hybridoma SFM™ (Gibco, Long Island, N.Y.) as described previously (Levesque et al. (2001) Leukemia, Vol. 15: 1307-1307; Levesque et al. (2003) Leukemia, Vol. 17: 442-450). All cultures are incubated at 37° C., 5% CO2 in air. After cells are plated, media containing 2 μM COG112 (SEQ ID NO: 2) or COG056 (an inactive control; SEQ ID NO: 3) is added and incubated for 3 hours. The cells are collected and washed prior to lysis in a Nonidet P-40 (NP40) lysis buffer containing protease and phosphatase inhibitors to prepare a clarified extract.

Immunoblotting is performed on the extracts from COG112 treated and untreated CLL cells to determine the relative levels of phospho-and total-(phosphorylated plus non-phosphorylated) ERK, Akt, IκK and NFκB. Specifically, cell extracts obtained by the method above will be analyzed to determine the protein concentration of each lysate using the BCA assay kit (Pierce). For immunoblotting, 30 μg of total protein lysate are loaded for each sample onto a 12.5% sodium dodecyl sulfate-polyacrylamide gel (SDS-PAGE) and electrophoresed using a Tris-Glycine SDS buffer (Bio-Rad). After electrophoresis, the proteins are electroblotted onto PVDF membranes (Bio-Rad). Membranes are blocked using 5% nonfat milk in Tris-buffered saline containing 0.1% Tween 20 (TBST) for 3 hours, washed with TBST and incubated overnight at 4° C. in a mouse anti-Akt antibody and a rabbit anti-phospho-Akt antibody (Cell Signaling). Membranes are washed with TBST for 1 hour with three changes of the wash solution and incubated with a donkey anti-rabbit antibody labeled with IRDye® 800 and a goat anti-mouse antibody labeled with IRDye® 680 (LiCor). Protein bands are visualized and quantitated using an Odyssey Infrared scanner (LiCor). This method allows for simultaneous quantitation of the phospho-Akt using the emission of the 800 nm channel and of the total Akt using the emission of the 680 nm channel. Following the initial read, a rabbit anti-β-Actin antibody is incubated with the membrane for 1 hour and the membrane is washed. A donkey anti-rabbit antibody labeled with IRDye® 800 is added and the blot read so that β-Actin signal at 800 nm is measured and can be used as a loading control to standardize the data. Similar methods are used for performing and analyzing immunoblots for phospho- and total-ERK, IκK and NFκB with the apporpriate matched pairs of antibodies. Quantitative analysis is completed by determining an activation index for each specific protein which is computed as the ratio of the specific phosphorylated protein quantity divided by the total phosphorylated plus non-phosphorylated specific protein multiplied by 100 to give a percentage of activation. Three samples are run for the B-CLL cells that are untreated, treated with COG112, or treated with COG056. COG112 treated CLL cells are expected to exhibit a significant reduction in the quantity of phosphorylated proteins relative to the untreated cells indicating a reduced activation of these signaling cascades.

### Example 5

### Effects of COG112 on CLL in Eμ-TCL1 Transgenic Mice

COG112 possesses a potent and selective cytotoxic activity against CLL cells isolated from human patients (Example 3). In the experiments outlined in this example, the effects of COG112 on CLL in Eμ-TCL1 transgenic mice are evaluated to determine if COG112 exhibits similar efficacy in vivo. Based on evidence that TCL1 is expressed in CLL cells but not normal mature B-cells and that TCL1 interacts with and enhances the activity of Akt, mouse models that demonstrate significant CLL pathology have been developed by targeted expression of TCL1. In the Eμ-TCL1 transgenic mouse model, the TCL1 gene is placed under control of a B-cell-specific IgVH promoter and IgH-Eμ enhancer. These mice develop normally into adulthood but later develop enlarged spleens, livers, and lymph nodes that are accompanied by high blood lymphocyte counts. The mice eventually die prematurely as the leukemic cells accumulate along with development of advanced lymphoadenopathy Importantly, the accumulated B-cells in these transgeneic TCL1 mice are G0-1 arrested and express CD19+/CD5+/IgM+ just as the human CLL cells (Bichi et al. (2002) Proc Natl Acad Sci USA, Vol. 99: 6955-6960). As further validation of these Eμ-TCL1 transgenic mice as an effective model of human CLL, CLL symptoms in the TCL1 transgenic mice improve following administration of fludarabine, a clinically used anti-CLL therapeutic. Fludarabine treatment improved the survival of mice, reduced the white cell counts and reduced the spleen size in treated animals relative to untreated controls given saline injections (Johnson et al. (2006) Blood, Vol. 108: 1334-1338). The effect of COG112 on CLL cell production and life expectancy in the Eμ-TCL1 transgenic mouse model of CLL is evaluated.

In a first series of experiments, twelve aged transgenic Eμ-TCL1 mice (9 to 12 months) demonstrating signs of leukemia are assigned to one of two treatment groups (vehicle control or COG112) based on white cell counts so that each group has a similar mean and range of white cell counts at the initiation of treatment. Animals (n=6) are treated with either 10 mg/kg of COG112 or a vehicle control daily by subcutaneous injection. After 15 days of treatment, blood is collected by retro-orbital bleeds and white bloods cells quantitated. White-blood cell counts are expected to be reduced in COG112-treated animals relative to the vehicle controls.

In a second series of experiments, aged transgenic Eμ-TCL1 mice (9 to 12 months) demonstrating signs of leukemia are randomly assigned to treatment groups (control, or one of three doses of COG112). At the initiation of treatment, blood is drawn to determine baseline CD5+/CD19+ CLL cell counts and groups of animals (n=20) are treated with a vehicle control (lactated Ringer's solution) or COG112 at doses of 4.0, 1.0, or 0.25 mg/kg. COG112 or the vehicle control is delivered by intraperitoneal injection at a volume of 10 mL/kg. Animals receive injections daily Monday through Friday so that 5 doses are administered per week for 5 weeks (35 days of total treatment). Disease course is monitored by survival, total blood leukocyte and lymphocyte count, and CD19+/CD5+ cell count weekly.

Blood is collected from each mouse on a weekly basis by retro-orbital bleeds for determination of total blood leukocyte and lymphocyte counts as well as CD19+/CD5+ cell counts to assess the leukemia burden. After 5 weeks (35 days of treatment), mice are euthanized and the post treatment leukemia burden is measured by cell counting, spleen weight, and histological analysis of bone marrow, spleen, liver, and lymph nodes. All mice dying before 35 days are analyzed in a comparable fashion. COG112 treatment is expected to produce as dose-dependent increase in cumulative survival and a dose-dependent reduction in CD19+/CD5+ cell counts, spleen weight, and CLL burden by histological analysis compared to vehicle-treated animals.

### Example 6

### Effect of COG Peptides on Breast Cancer Cell Lines

As shown in Example 3, ApoE peptides are cytotoxic to cancerous B cells. Here, we have designed experiments to show that COG peptides are effective in other types of cancers, such as breast cancer, that are associated with aberrant cellular signaling. The effect of COG peptides on PI-3K/Akt signaling pathways and cell growth in three different breast cancer cells is evaluated.

Numerous breast cancer cell lines exist that have well documented activity for Estrogen Receptor, Akt activation status, Her2 expression and PP2A activity. Three cell lines have been selected for analysis that represent both ER positive and negative tumors as well as Her2/Neu positive and negative lines (Table II). Published studies indicate that Akt, IκK and NFκB are activated in breast cancer cell lines leading to an anti-apoptotic state. To investigate the effect of ApoE peptides on the signaling cascades in these cell lines, growth curves are analyzed and signal transduction related proteins are evaluated using immunoblotting to determine the relative levels of phospho- and total-(phosphorylated plus non-phosphorylated) Akt, IκK and NFκB.

TABLE II
Breast Cancer Cell Lines Used for Analysis

| | ER | Akt | | PP2A |
|---|---|---|---|---|
| Cell Line | Status | Activation | Her2/Neu | Expression |
| MCF7 | + | + | − | ↓ |
| MDA-MB-231 | − | + | − | ↓ |
| BT-474 | − | + | + | |

The cell lines described above are cultured in the ATCC recommended media in 48 well plates. After cells are plated, media containing one of the following COG peptides or an inactive peptide control is added and cells are incubated at 37° C., 5% CO2 in air. The COG peptides are tested at a range of concentrations to obtain a dose titration for growth inhibition. Cultures are analyzed for growth daily using the tetrazolium compound 3-(4,5-dimethylthiazol-2-yl)-5-(3-carboxymethoxyphenyl)-2-(4-sulfophenyl)-2H-tetrazolium, inner salt (MTS) and the CellTiter 96® AQueous One Solution Cell Proliferation Assay from Promega (Madison, Wis.).

| | | |
|---|---|---|
| COG133 | LRVRLASHLRKLRKRLL | (SEQ ID NO: 1) |
| COG112 | RQIKIWFQNRRMKWKKCLRVRLASHLRKLRKRLL | (SEQ ID NO: 2) |
| COG1410 | AS(Aib)LRKL(Aib)KRLL | (SEQ ID NO: 4) |
| | [Aib is amino isobutyric acid] | |
| COG345 | LRVRLAS-aib-LRKLRK(ac)RLL | (SEQ ID NO: 6) |
| | [Aib is amino isobutyric acid and ac is acetyl lysine] | |
| COG056 | LLRKRLKRLHSALRVRL | (SEQ ID NO: 3) |
| (inactive control) | | |
| COG095 | RQIKIWFQNRRMKWKKC | (SEQ ID NO: 7) |
| (inactive control) | | |

Following determination of growth curves, a new batch of cells is plated, media containing one of the above-listed COG peptides (COG133, COG112, COG1410, or COG345) or inactive peptide controls (COG056 or COG095) is added, and incubated with cultures for various time periods. The cells are collected, washed, and lysed in a Nonidet P-40 lysis buffer containing phosphate buffered saline with a dissolved Complete protease inhibitor tablet (Boehringer Ingleheim) and phosphatase inhibitors (Roche). Lysates are centrifuged at 20,000×g for 20 min at 4° C. The supernatants are transferred to fresh tubes and the protein concentrations of the lysates determined using the BCA assay kit (Pierce). Immunoblots for phospho- and total-Akt, IκK and NFκB are performed according to the methods described in Example 4. It is expected that breast cancer cells treated with an active COG peptide will exhibit a reduction in growth rate and a significant decrease in the quantity of phosphorylated proteins as compared to untreated cells or cells treated with inactive control peptides.

### Example 7

### Effects of ApoE Peptides on Breast Cancer Tumor Growth

Xenograft models for each breast cancer cell line (MDA-MB-231, MCF-7 or BT-474) are used to determine the effect of COG peptide treatment of tumors in vivo. Tumor fragments (30 to 70 mg) are implanted into female outbred nude mice (NIHIII for the BT474 cell line) high in the right axilla of 180-240 animals per tumor type. After several days, mice are triaged and distributed into treatment groups when mean tumor burden is ˜125 mg so that each group is within 10% of the overall mean initial tumor burden. Mice are treated (n=20) daily by i.p. injection with either a vehicle control, a negative control peptide (COG056), a positive control (paclitaxel, Gemcitabine or Lapatinib), or one of 3 doses (0.25, 1.0, or 4.0 mg/kg) of an ApoE peptide (e.g. COG112, COG133, COG1410, and COG345). In mice implanted with cells from the MCF7 cell line, estradiol pellets are implanted weekly. Body weights and tumor sizes are recorded twice a week and clinical signs are monitored daily. Animals are treated until tumor burdens reach 1 g for Tumor Growth Delay Endpoint and complete regression/partial regression/tumor free survivor determination. Treatment with the ApoE peptides (e.g. COG peptides) is expected to significantly slow tumor growth over time as compared to vehicle or negative control peptide treatment.

### Example 8

### COG112 Inhibits Growth of K562 CML Cells

Chronic myelogenous leukemia (CML) is characterized by progression from the indolent chronic phase (CP) to the aggressive myeloid or lymphoid blast phase (BP) phase (Faderl et al. (1999) N. Engl. J. Med., Vol. 341: 164-172) that is biologically similar to acute leukemia. Emergence and maintenance are dependent on the unrestrained kinase activity of BCR/ABL oncoproteins (Van Etten et al. (1989) Cell, Vol. 58: 669-678; McLaughlin et al. (1987) Proc. Natl. Acad. Sci. USA, Vol. 84: 6558-6562). This constitutive activity of these oncoproteins recruit and activate multiple pathways, such as the PI-3K/Akt pathway, that transduce oncogenic signals, leading to increased survival, enhanced proliferation, and arrested differentiation of hematopoietic progenitors (See FIG. 1A; Elefanty et al. (1990) EMBO J, Vol. 9:1069-1078).

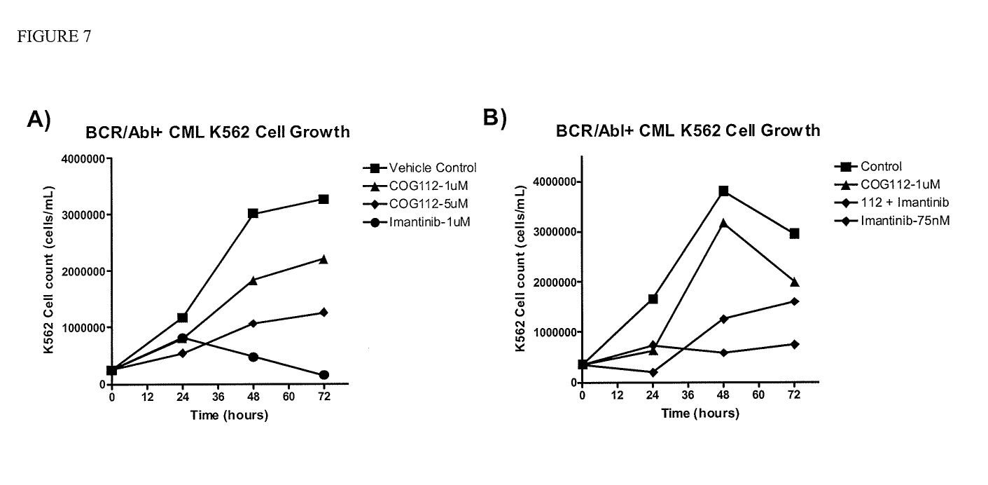

Since ApoE peptides can modulate the PI-3K/Akt signaling pathway (Example 1), which is constitutively activated in CML, and the ApoE peptides effectively killed CLL cells (Example 3), we evaluated the effect of COG112 on the BCR/Abl+ K562 CML cell line to determine if COG112 could slow the growth of K562 cells. Cells (20 mL) were seeded in a T-75 flask at a concentration of 0.25×10⁶ cells/mL. Each day after seeding, 0.20 mL of cell suspension was removed and centrifuged to collect cells. The cell pellet was resuspended in 500 μL of serum free media and 100 μL of trypan blue stain was added and incubated for 5 minutes before counting the trypan blue positive cells with a hemacytometer. COG112 produced a dose dependent inhibition of K562 cell growth (FIG. 7A). Imatinib (Gleevec, 1 μM), used as a positive control, significantly reduced cell numbers. To further extend the characterization of COG112 and to determine if the activity of COG112 was synergistic or antagonistic with the activity of Imatinib, a similar growth analysis was performed with reduced concentrations of COG112 and Imatinib. Treatment with 1 μM COG112 or 75 nM Imatinib produced suboptimal inhibition of K562 cell growth (FIG. 7B). However, when the two treatments were combined, the cell growth was inhibited to a greater extent than either treatment alone indicating that the treatments produced an additive effect. These data demonstrate that ApoE peptides effectively reduce growth of CML cancer cells and may provide an effective therapeutic alone or in combination with other drugs for the treatment of CML. Furthermore, ApoE peptides may provide a novel therapeutic approach for the treatment of imatinib/dastinib resistant CML, CML-BC and Ph1(+) ALL.

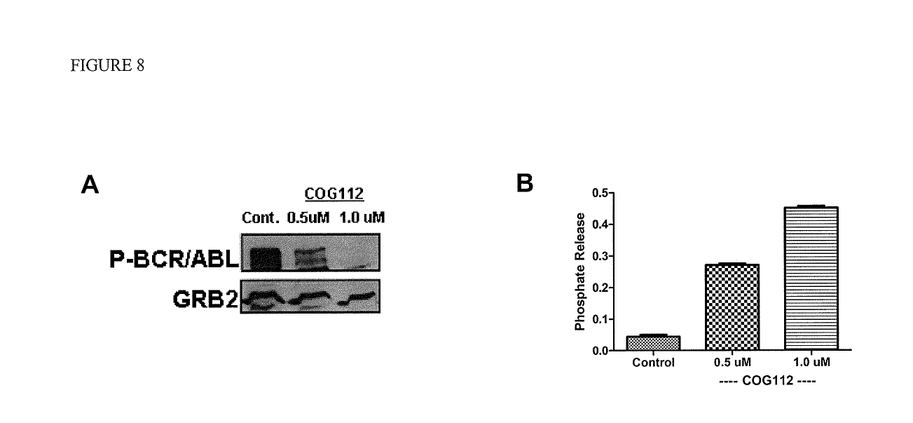

To further characterize the mechanism by which COG112 produced cytotoxicity of BCR/Abl+ K562 CML cells, the effect of COG112 on phosphorylated BCR/ABL fusion protein and PP2A activity in K562 CML cells was determined. As discussed above, constitutive activation of the BCR/ABL fusion protein produced by the Philadelphia chromosome (e.g. product of the t(9;22)(q34;q11) translocation) is the primary causative factor in the persistent signaling that leads to unrestrained cell proliferation and survival in CML. To determine whether COG112 had an effect on BCR/ABL activation, K562 cells were left untreated or treated with COG112 at doses of 0.5 or 1 μM for 24 hours. Cell lysates were prepared and subject to Western blot analysis to detect activated phospho-BCR/ABL. COG112 treatment of K562 cells produced a dose-dependent reduction in the level of phosphorylated BCR/ABL (FIG. 8A). To test whether the effect of COG112 on BCR/ABL activation could be due to activation of PP2A, BCR/ABL+ K562 CML cells were treated for 24 hours with COG112 (0.5 μM or 1 μM) and PP2A activity was measured using an immunoprecipitation/phosphate release assay as described previously (Neviani et al. (2007) J. Clin. Invest., Vol. 117: 2408-2421). A robust activation of PP2A was observed in K562 cells upon treatment with COG112 (FIG. 8B). Taken together, these results suggest that ApoE peptides, such as COG112, can reduce the activation of the BCR/ABL oncogene through the enhancement of PP2A activity and thereby reduce the growth of BCR/ABL+ K562 CML cells. Such a mechanism of action would be particularly useful in treating imatinib/dastinib resistant BCR/ABL+ cancers where PP2A activation has been reported to inhibit the growth of imatinib/dastinib resistant cell lines (Neviani et al. (2007) J. Clin. Invest., Vol. 117: 2408-2421).

### Example 9

### COG112 Inhibits Growth of Jurkat T-Cell Leukemia Cells

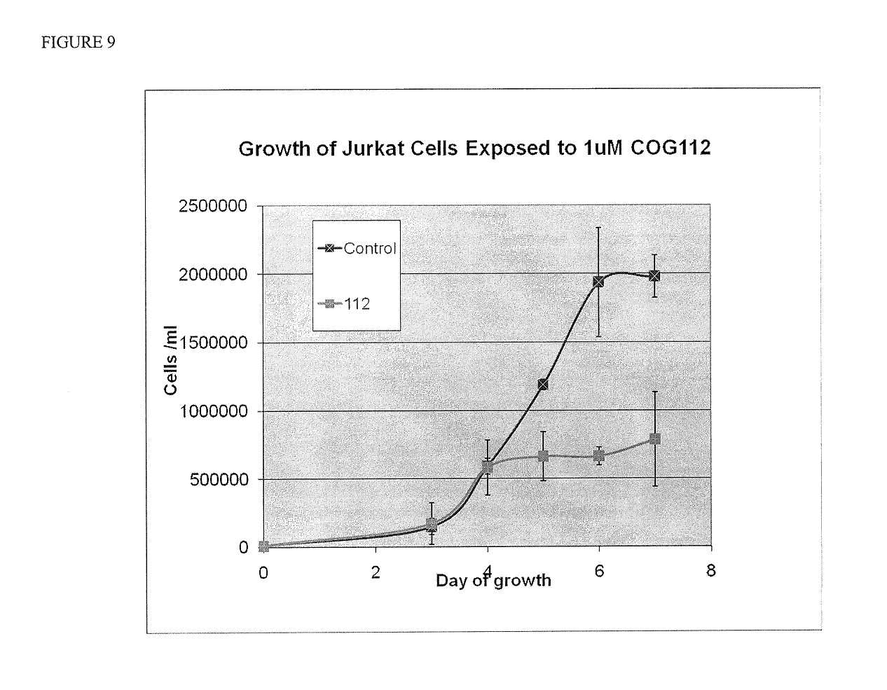

To determine whether ApoE peptides could be therapeutic for T-cell leukemia, the effect of COG112 on the growth rate of Jurkat T-cells, a human acute T-cell leukemia cell line, was assessed. Jurkat T-cells (2×10⁴ cells per well in a 6 well plate) were grown in 2 mL of media in the absence or presence of 1 μM COG112 added upon plating the cells. Cell growth was measured using a hemacytometer following daily removal of a small aliquot for counting. As shown in FIG. 9, COG112 significantly inhibited the proliferation of the T-cells, suggesting that ApoE peptides may also be therapeutic for T-cell leukemias as well as B-cell leukemias.

It is understood that the disclosed invention is not limited to the particular methodology, protocols and reagents described as these may vary. It is also understood that the terminology used herein is for the purposes of describing particular embodiments only and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to “the antibody” is a reference to one ore more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the exemplary methods, devices, and materials are as described. All patents, patent applications and other publications cited herein and the materials for which they are cited are specifically incorporated by reference in their entireties.

Those skilled in the art will recognize, or be able to ascertain using no more than routine experimentation, many equivalents to the specific embodiments of the invention described herein. Such equivalents are intended to be encompassed by the following claims.

1. A method of treating cancer in a subject in need thereof comprising administering an effective of amount of at least one ApoE peptide to the subject.

2. The method of claim 1, wherein administration of said ApoE peptide reduces at least one symptom of cancer in the subject.

3. The method of claim 1, wherein administration of said ApoE peptide increases PP2A activity in a cancer cell of the subject.

4. The method of claim 1, wherein administration of said ApoE peptide decreases Akt kinase, IκK kinase, or NFκB activity in a cancer cell of the subject.

5. The method of claim 1, wherein said ApoE peptide contains 17 amino acids or more.

6. The method of claim 1, wherein said ApoE peptide contains 20 amino acids or more.

7. The method of claim 1, wherein said ApoE peptide contains 30 amino acids or more.

8. The method of claim 1, wherein said ApoE peptide contains 40 amino acids or more.

9. The method of claim 1, wherein said ApoE peptide contains the sequence of SEQ ID NO: 1.

10. The method of claim 1, wherein said ApoE peptide is conjugated to a protein transduction domain.

11. The method of claim 10, wherein said protein transduction domain is selected from the group consisting of peptides derived from antennapedia, TAT, SynB1, SynB3, SynB5, and polyarginine.

12. The method of claim 11, wherein said ApoE peptide contains the sequence of SEQ ID NO: 2.

13. The method of claim 1, wherein said cancer is leukemia.

14. The method of claim 13, wherein said leukemia is chronic lymphocytic leukemia (CLL).

15. The method of claim 14, wherein administration of said ApoE peptide decreases the number of CD5+ B cells in the subject.

16. The method of claim 13, wherein said leukemia is chronic myelogenous leukemia (CML).

17. The method of claim 16, wherein administration of said ApoE peptide decreases the growth of BCR/ABL+ cells in the subject.

18. The method of claim 17, wherein the BCR/ABL+ cells are resistant to imatinib or dasatinib.

19. The method of claim 13, wherein said leukemia is acute lymphocytic leukemia (ALL).

20. The method of claim 1, wherein said cancer is breast cancer.

21. The method of claim 20, wherein said breast cancer is characterized by Her2 expression.

22. The method of claim 20, wherein said breast cancer is characterized by estrogen receptor expression.
